(19) United States
(12) Patent Application Publication
(10) Pub. No.: US 2017/0140134 A1
(43) Pub. Date: May 18, 2017

(54) MEDICAL DEVICE USER CACHING

(71) Applicant: Welch Allyn, Inc., Skaneateles Falls, NY (US)

(72) Inventors: Stacie L. Brough, Syracuse, NY (US); Andrew Brayton Nye, III, Skaneateles, NY (US)

(21) Appl. No.: 14/941,843

(22) Filed: Nov. 16, 2015

(51) Int. Cl. G06F 21/31; G06F 19/00; A61B 5/00; G06F 12/08

(52) U.S. Cl. CPC G06F 21/31; G06F 12/0875; G06F 19/3418; A61B 5/0002; G06F 2212/45; G06F 2212/1052

(57) ABSTRACT

An example medical device includes a physiological measurement device, a device management engine, a user caching engine, and a login engine. The device management engine is configured to receive data captured by the physiological measurement device. The user caching engine is configured to store cache records associated with users in a user cache. The login engine is configured to receive a user identifier associated with a user and determine whether the user identifier is associated with a cache record stored in the user cache. When determined that the user identifier is associated with a cache record stored in the user cache, the login engine is configured to log the user in. When not determined that the user identifier is associated with an unexpired cache record stored in the user cache, the login engine is configured to prompt the user for a credential.

### INTRODUCTION

Medical devices may be used in healthcare environments, such as hospitals and clinics, to measure and monitor physiological parameters of patients. For example, some medical devices may be used to measure vital signs of a patient. Health information systems are also used in healthcare environments to record various information about patients. Health information systems may store biographical data about patients and historical records related to the patients. Health information systems may also store data associated with measurements of physiological parameters captured using medical devices, including user identification data for a healthcare practitioner using the medical device. Healthcare environments often include other servers as well, such as user authentication servers.

### SUMMARY

In one aspect, a medical device, comprising: a physiological measurement device; a device management engine configured to receive data captured by the physiological measurement device; a user caching engine configured to store cache records associated with users in a user cache; and a login engine configured to: receive a user identifier associated with a user; determine whether the user identifier is associated with a cache record stored in the user cache; and when determined that the user identifier is associated with a cache record stored in the user cache, log the user in.

In another aspect, a method of logging a user into a medical device, the method comprising: receiving, by the medical device, a user identifier associated with the user; determining whether the user identifier is associated with a valid cache record stored in the user cache; when determined that the user identifier is associated with a valid cache record stored in the user cache, logging the user in; and when determined that the user identifier is not associated with a valid cache record: prompting the user for a credential; determining if the user is authenticated based on the user identifier and credential; when determined that the user is authenticated: adding a record associated with the user to the user cache; and logging the user in.

In yet another aspect, A medical device, comprising: a physiological measurement device; a device management engine configured to receive data captured by the physiological measurement device; a user caching engine configured to store cache records associated with users in a user cache; and a login engine configured to: receive a user identifier associated with a user; determine whether the user identifier is associated with an unexpired cache record stored in the user cache; when determined that the user identifier is associated with an unexpired cache record stored in the user cache: log the user in; update a last login time of the cache record associated with the user identifier. when not determined that the user identifier is associated with an unexpired cache record stored in the user cache: prompt the user for a password; send an authentication request to a server device; receive a response to the authentication request; and when determined that the authentication request was successful, add a record associated with the user to the user cache.

### DESCRIPTION OF THE FIGURES

FIG. 1 shows a block diagram of an example system for communicating between medical devices and servers.

FIG. 2 illustrates an example medical device of FIG. 1.

FIG. 3 illustrates another example medical device of FIG. 1.

FIG. 4 illustrates a block diagram of an example medical device of FIG. 1.

FIG. 5 illustrates an example process to login a user performed by embodiments of the medical devices of FIG. 1.

FIG. 6 illustrates an example table of cache records for user information.

FIG. 7 is an example communication diagram between the user, the medical device, and the server of FIG. 1.

FIG. 8 illustrates an example process to maintain a user cache performed by some embodiments of the medical devices of FIG. 1.

FIG. 9 illustrates an example screen for user logins generated by some embodiments of the interface engine of FIG. 4.

FIG. 10 illustrates another example screen for user logins generated by some embodiments of the interface engine of FIG. 4.

FIG. 11 illustrates an example screen for user login settings generated by some embodiments of the interface engine of FIG. 4.

FIG. 12 illustrates example components of a medical device of the system of FIG. 1.

### DETAILED DESCRIPTION

Medical devices may be used to capture various physiological measurements or readings from a patient. For example, a medical device may measure one or more of the body temperature, blood pressure, heart rate, and blood oxygen content as well as many other types of physiological parameters.

Before capturing readings, a caregiver (e.g., nurse, doctor, etc.) may be required to login to the medical device. The login information may be used to access a system external to the medical device. For example, it may be beneficial to store a measurement of a physiological parameter of a patient captured by a medical device in an electronic health record associated with the patient that is stored on a health information system (HIS) server. Additionally, it may be beneficial to alert a billing server of various measurements that have been taken using a medical device in order to facilitate billing and collecting payments for the measurement. The foregoing are just examples. There are, of course, many other servers that perform many other functions with which it may be beneficial for a medical device to communicate.

In at least some of these situations, the medical device performs a user login process. For example, the login process may authenticate the user locally. Or the login process may facilitate logging in to a remote server such as a health information system server or an authentication server. For example, the medical device may receive identification information (e.g., a username) and a credential (e.g., a password) from the user. The medical device may then transmit the username and password to an authentication server for authentication. If the authentication server indicates that the authentication was successful, the medical device allows the user to capture physiological parameters. In addition to an indication that the authentication was completed successfully, the authentication server may provide additional information to the medical device such as another identifier associated with the user that is used by the authentication server or another system (e.g., a health information system such as an electronic medical record system).

On the other hand, if the authentication is not successful, the medical device may invoke a procedure for handling failed authentication attempts. For example, in some embodiments, the user will be given additional chances to correctly authenticate, but after a predetermined number of unsuccessful authentication attempts, the user account will be locked.

Entering credential information on the medical device may be challenging or tedious for the user. The input devices on some medical devices may make it difficult to enter a password. For example, some medical devices include smaller display screens that are touch sensitive and display virtual keyboards for entering character information such as passwords. Because of the smaller screen size, the region of the touch-sensitive region of the screen for each character may be quite small, which can lead to users frequently touching incorrect characters while entering passwords. This problem may be exacerbated as users of medical devices may be wearing gloves, which may decrease dexterity and further increase the likelihood of touching the wrong character. Additionally, users may not realize when they have entered the wrong character in a password as the input data may be obscured to prevent viewing by others. As password requirements (e.g., length, special characters, etc.) become more stringent, these challenges grow. Additionally, in a medical environment it may be common for multiple users to share a medical device throughout the day. Thus users may need to frequently re-login to the medical device.

In some embodiments, the medical device caches user identification information after successful logins. The medical device can then allow a user whose identification information is stored in the cache to access and use the medical device without completing a full login process. For example, a cached user may not be required to enter a password. Instead, upon entering user identification information, the medical device may determine that the user identification is stored in the cache and permit the user to access the medical device. In at least some embodiments, the cached user information includes a user identification but does not include a credential associated with that user identification (e.g., a password). In this manner, user's passwords are not stored on the medical device. However, in some embodiments, a user's password may be stored with or without encryption in the cache so that the password may later be provided to the authentication server to reauthenticate the user while the user is cached.

In various embodiments, various policies are used to manage the user identification information stored in the cache. For example, in some embodiments, cached user identification information expires after a pre-determined time period has elapsed since the associated user last logged in or logged out of the medical device. Examples of the pre-determined time period are one hour, two hours, four hours, and eight hours. Other embodiments may use other time periods as well. Alternatively, or additionally, the cached user identification information may expire after a pre-determined number of logins. For example, in some embodiments, the predetermined number of logins is two, three, four, five, ten, or another number of logins.

As another example, cached user information may expire at one or more pre-determined times of the day. For example, the user information may expire at one or more times of each day that are associated with the end of work shifts within the healthcare environment. In some embodiments, cached user information expires based on a combination of pre-determined times of the day and an elapsed time since a user last logged in. For example, cached user information may expire if it is both after nine o'clock (i.e., an example pre-determined time of the day) and it has been at least an hour (i.e., an example elapsed time) since the associated user last logged in to the medical device. In this manner, the settings for causing cached user information to expire may be configured to support users with overlapping shifts.

As yet another example, only a predetermined number of users are cached in some embodiments. For example, the predetermined number may be one, two, five, ten, or twenty. Other embodiments use other predetermined numbers as well. In these embodiments, when the predetermined number of users has been reached, the medical device may remove user information from the cache before storing new user information. In some embodiments, the user information associated with the one user with the oldest last login or last logout time is removed in order to store user information associated with one new user. Beneficially, by limiting the number of users that are cached, the amount of memory required for the user cache can be limited.

Further, in some embodiments, a user can perform a manual logout action (e.g., by selecting a logout option) that removes the user from the cache. For example, the user can logout at the end of a shift or before leaving the clinic for a period of time. Additionally, in some embodiments, a user (such as an administrator) can clear all of the users in the cache. For example, a user may clear the user cache when the medical device is moved from one department to another.

In some embodiments, the medical device includes global settings that apply to caching all users. For example, the pre-determined time period for user information to expire or the pre-determined times of the day when user information expires may be defined in global settings that are applicable to all users. Additionally, in some embodiments, the medical device may apply per-user settings or role-based settings for these parameters. For example, user information associated with a user with the role “doctor” may expire after a first predetermined time period, while user information associated with a user with the role “nurse” may expire after a second predetermined time period. Similarly, the roles may be associated with different times of day at which the cached user information expires. These role-based settings may be based on typical staff schedules within the healthcare environment. Further, in some embodiments, the authentication server may define expiration settings globally or for individual users. For example, the authentication server may transmit expiration settings for an individual user to the medical device in response to a successful authentication attempt. These settings specified by the authentication server may override settings on the medical device. For example, the authentication server may indicate that a particular user should be blacklisted from the cache and thus should never be cached. Conversely, the authentication server may indicate that a particular user should be whitelisted so that the user remains in the cache indefinitely. Additionally, in some embodiments, a whitelist or blacklist of users can be setup locally on the medical devices 102, 104 by an administrator or other user.

The medical devices may use various techniques to remove cached user information that has expired. For example, in some embodiments, a cache maintenance process runs on a schedule or at various intervals (e.g., every five minutes, every fifteen minutes, every hour, etc.) to evaluate the cached user information and remove information that has expired according to the policies that are currently in place. In other embodiments, the cached user information is evaluated against the policies at the time the associated user attempts to login again. If the cached user information is still valid (e.g., it has not expired) according to the policies, the user will be permitted to login without authentication. If not, the user information will be removed from the cache and re-added upon a successful authentication.

In at least some embodiments, the caching of user information is performed entirely on the medical device. Beneficially, the medical device with user caching can be used in an existing healthcare environment with existing authentication servers and health information systems without changing the authentication servers or health information systems. In other words, some embodiments of the medical device with user caching are backwards compatible with existing healthcare environments.

Beneficially, the medical device with user caching allows a group of users such as the caregivers that might use the medical device during a typical shift in a healthcare environment to conveniently access and share the medical device without having to reenter a password each time a different user starts to use the device.

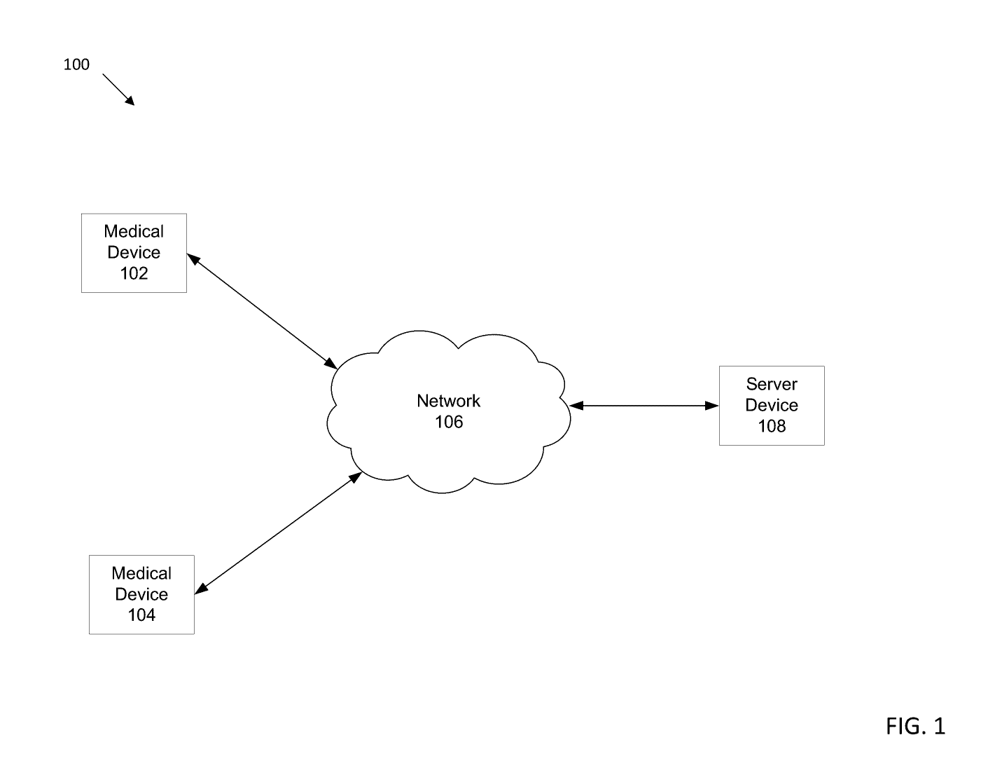

FIG. 1 is a block diagram illustrating an example system 100 for capturing physiological parameters with a medical device that caches user information. The system 100 includes medical devices 102 and 104, network 106, and server device 108.

In this example, the medical devices 102, 104 are used to collect physiological data from patients. As used herein, “medical device” refers to any device that can be used in a medical environment. Examples of medical devices include, but are not limited to, thermometers, blood pressure sensors, heart rate sensors, pulse rate sensors, SpO2 sensors, devices to monitor vital signs, devices for measuring body weight, devices for metering fluids, etc. These medical devices can be located in a medical environment. Medical environments, such as clinics, hospitals, nursing homes, care facilities, and at-home care stations, can rely on peripheral devices that communicate, display, and coordinate the monitoring and maintenance of a patient's health. In one example, the medical devices 102, 104 are located in the same medical environment. In another example, the devices are located in different medical environments that are spread out geographically.

The medical devices 102, 104 communicate using the network 106. In one example, the medical devices 102, 104 and the network 106 are part of a CONNEX™ system from Welch Allyn of Skaneateles Falls, N.Y., although other systems can be used. In such an example, the medical devices 102, 104 communicate through known protocols, such as the Welch Allyn Communications Protocol (WACP). WACP uses a taxonomy as a mechanism to define information and messaging. Taxonomy can be defined as description, identification, and classification of a semantic model. Taxonomy as applied to a classification scheme may be extensible. Semantic class-based modeling utilizing taxonomy can minimize the complexity of data description management by limiting, categorizing, and logically grouping information management and operational functions into families that contain both static and dynamic elements.

The network 106 is an electronic communication network that facilitates communication between the medical devices 102, 104 and the server device 108. An electronic communication network is a set of computing devices and links between the computing devices. The computing devices in the network use the links to enable communication among the computing devices in the network. The network 106 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various embodiments, the network 106 includes various types of links. For example, the network 106 can include wired and/or wireless links. Furthermore, in various embodiments, the network 106 is implemented at various scales. For example, the network 106 can be implemented as one or more local area networks (LANs), metropolitan area networks, subnets, wide area networks (such as the Internet), or can be implemented at another scale.

In this example, the medical devices 102, 104, and the network 106 are all part of the same network. In other words, the medical devices 102, 104 and the network 106 communicate with one another over a LAN behind a wall safeguarding the devices from outside influences on the Internet, such as a firewall.

The medical devices 102, 104 can provide various types of functionality, including measuring or monitoring patient physiological parameters. The medical devices 102, 104 can include one or more physiological monitor devices configured to measure and possibly display representations of one or more physiological parameters of a patient. In addition, the medical devices 102, 104 can include one or more desktop, laptop, or wall-mounted devices. In some embodiments, the medical devices 102, 104 are configured to be used by a caregiver to monitor the physiological parameters of multiple patients at one time. Such monitor devices are typically not wall mounted.

The medical devices 102, 104 can communicate with each other through the network 106. In various embodiments, the medical devices 102, 104 can communicate various types of data with each other through the network 106. For example, in embodiments where the medical devices 102, 104 include a set of physiological monitor devices and a separate monitor device, each of the physiological monitor devices can send data representing measurements of physiological parameters of patients to the monitor device. In this way, the medical devices 102, 104 can display representations of physiological parameters to a caregiver.

The medical devices 102, 104 can send various types of data and can receive various types of data through the network 106. For example, in some embodiments, the medical devices 102, 104 can send measurements of physiological parameters. In another example, the medical devices 102, 104 can retrieve past measurements of physiological parameters of patients.

The server device 108 communicates through the network 106 with the medical devices 102, 104. In this example, the server device 108 receives, stores, and associates physiological parameters measured by the medical devices 102, 104 with patient records. In some embodiments, the server device 108 includes a health information system, such as a system that captures, stores, manages or transmits information related to the health, including healthcare information, of individuals or organizations that provide health and healthcare services. Examples of health information systems include electronic medical records (EMR) systems and electronic health record (EHR) systems. In some embodiments, the server communicates through known protocols, such as the Welch Allyn Communications Protocol (WACP). Additionally, in some embodiments, the server device 108 includes and authentication system that authenticates users of the medical devices 102, 104.

In some embodiments, the server device 108 is located “in the cloud.” In other words, the server device 108 is located outside of the internal network associated with the medical devices 102, 104. In such examples, the server device 108 may not communicate directly with the medical devices 102, 104 but may instead communicate with a central server located within the same network as the medical devices 102, 104 such as the CONNEX™ system from Welch Allyn of Skaneateles Falls, N.Y. Intermediary servers in the CONNEX™ system, in turn, communicate with the medical devices 102, 104. Alternatively, the medical devices 102, 104 communicate directly with the server device 108 even if the server is located outside of the internal network associated with the medical devices 102, 104.

The medical devices 102, 104 and the server device 108 include computing systems. As used herein, a computing system is a system of one or more computing devices. A computing device is a physical, tangible device that processes data. Example types of computing devices include personal computers, standalone server computers, blade server computers, mainframe computers, handheld computers, smart phones, special purpose computing devices, and other types of devices that process data. In addition to the computing systems, the medical devices 102, 104 may include special purpose components that operate to capture various physiological or other parameters. Further, the computing systems included in the medical devices 102, 104 may include special-purpose components that are configured to interact with the special-purpose components.

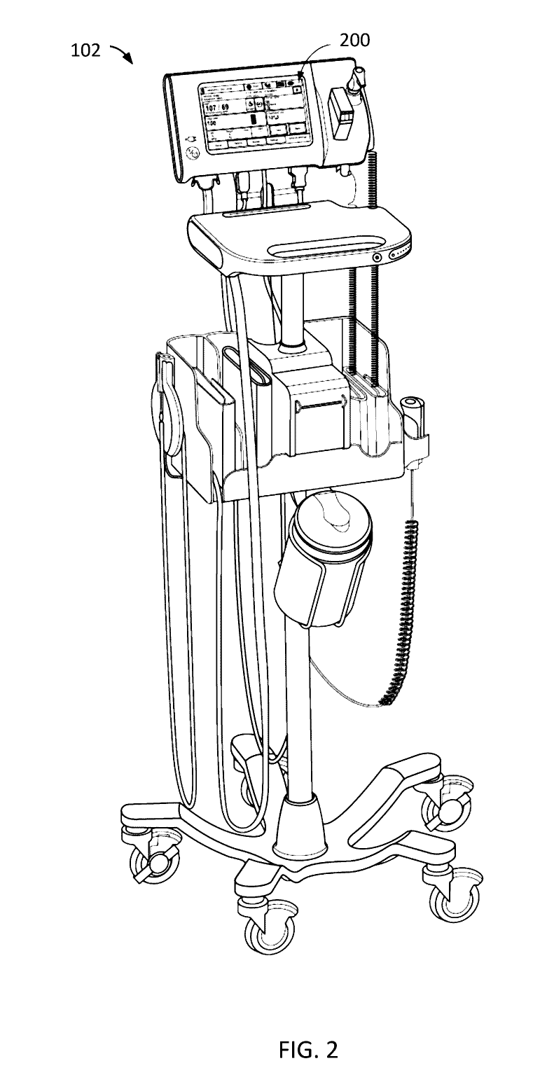

FIG. 2 illustrates one example of the medical device 102. The medical device 102 is shown on a mobile cart, and the medical device 102 is programmed to provide the functionalities described herein. The medical device 102 includes a display screen 200 and one or more physiological measurement devices that are configured to measure one or more physiological parameters of a health-care recipient, also referred to herein as a patient. The display screen 200 operates to display information. In some embodiments, the display screen 200 is a touch-sensitive display screen and operates to receive input from users. Some embodiments include additional or alternative input devices for receiving input from users. Other embodiments can include more or fewer components than those shown in FIG. 2, or can include different components that accomplish the same or similar functions.

The medical device 102 is able to operate within one or more profiles. A profile is a series of one or more tasks that a user of the medical device 102 performs. When the medical device 102 operates within a profile, the medical device 102 provides functionality suitable for assisting the user in performing the profile. When the medical device 102 operates within different profiles, the medical device 102 provides different functionality.

When the medical device 102 is manufactured, the medical device 102 is configured to be able to operate within one or more profiles. After the medical device 102 is manufactured, the medical device 102 can be reconfigured to operate within one or more additional profiles. In this way, a user can adapt the medical device 102 for use in different profiles as needed.

In various embodiments, the medical device 102 operates within various profiles. For example, in some embodiments, the medical device 102 can operate within an interval profile or a non-interval profile. Example types of non-interval profiles include, but are not limited to, a spot check profile and an office profile.

In example embodiments, the names for the profiles can be defined by the user. For example, the user can rename an “interval profile” as “ED 3 North” or any other nomenclature as desired to provide more context to the user.

When the medical device 102 is operating within the interval profile, the medical device 102 obtains a series of measurements of one or more physiological parameters of a single monitored patient over a period of time. In addition, the medical device 102 displays, on the display screen 200, an interval profile home screen. The interval profile home screen contains a representation of a physiological parameter of the monitored patient. The representation is based on at least one measurement in the series of measurements. A representation of a physiological parameter is a visible image conveying information about the physiological parameter.

For example, when the medical device 102 is operating within the interval profile, the medical device 102 can obtain a blood pressure measurement of a single patient once every ten minutes for six hours. In this example, the medical device 102 displays an interval profile home screen that contains a representation of the patient's blood pressure based on a most recent one of the blood pressure measurements. In this way, a user of the medical device 102 can monitor the status of the patient.

When the medical device 102 is operating within a non-interval profile, the medical device 102 obtains a measurement of one or more physiological parameters from each patient in a series of patients. In addition, the medical device 102 displays a non-interval profile home screen on the display screen 200. The non-interval profile home screen contains a representation of the physiological parameter of a given patient in the series of patients. The representation is based on the measurement of the physiological parameter of the given patient.

In one example, when the medical device 102 is operating within a spot check profile, the medical device 102 obtains blood pressure measurements from a series of previously-identified patients. In this other example, the medical device 102 displays a spot check profile home screen containing a blood pressure measurement of a given patient in the series of previously-identified patients. In this way, a user of the medical device 102 can perform spot checks on the blood pressures of patients who have already been admitted to a hospital.

As used in this document, a patient is a previously identified patient when the medical device 102 stores information regarding the identity of the patient. In another example, when the medical device 102 is operating within an interval profile, the medical device 102 can obtain a single blood pressure measurement from each patient in a series of unidentified patients as the patients arrive at a hospital.

The interval profile home screen may be different than the non-interval profile home screen. Further, as discussed below, the navigation options associated with the different profiles allow for efficient monitoring based on the environment in which the device is used. In various embodiments, the interval profile home screen is different than the non-interval profile home screen in various ways. For example, in some embodiments, the interval profile home screen includes at least one user-selectable control that is not included in the non-interval profile home screen. In other embodiments, a representation of a physiological parameter in the interval profile home screen has a different size than a representation of the same physiological parameter in the non-interval profile home screen.

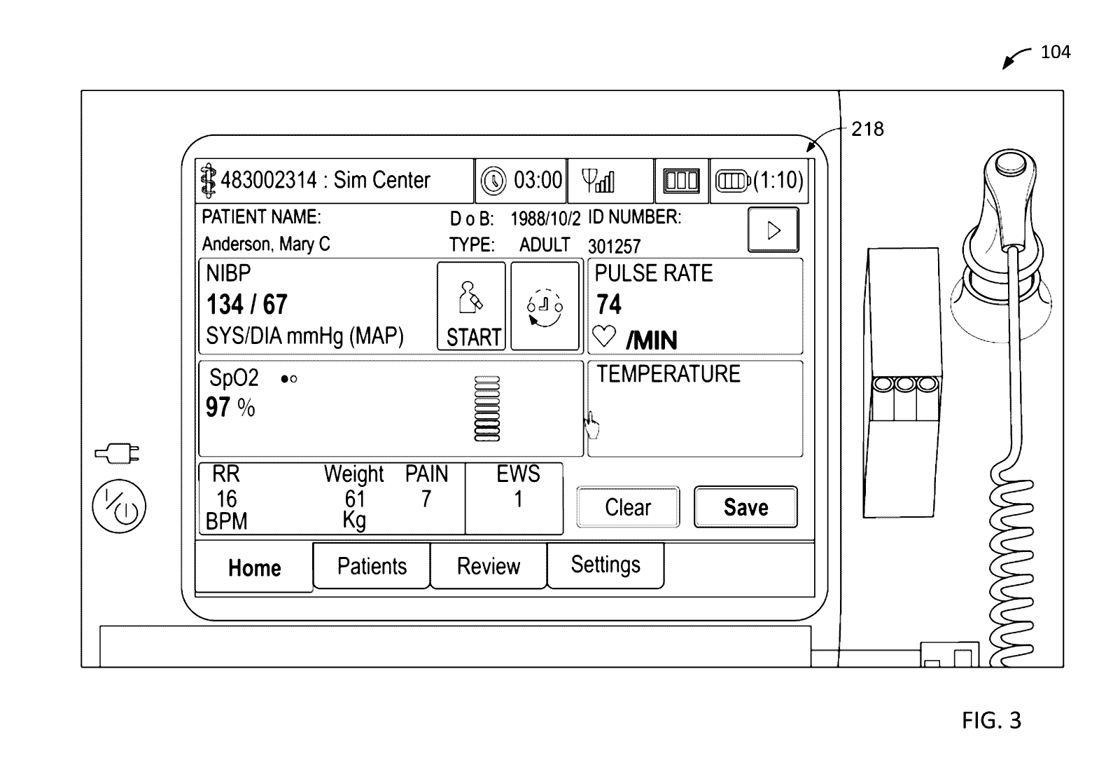

FIG. 3 illustrates one example of the medical device 104. In this example, the medical device 104 is similar to the medical device 102 described above. The medical device 104 is configured to be mounted on a wall and may also be programmed in a manner similar to that described above to monitor physiological parameters of a patient. Additionally, in some embodiments, the medical devices 102, 104 are stand-alone devices, which can mean that the medical devices 102, 104 are not part of a mobile cart or wall-mounted station.

In the examples described herein, the medical devices 102, 104 are computing devices that have been programmed to perform special, complex functions. These specially-programmed devices function to capture physiological measurements and transmit those physiological measurements to various external servers with greater efficiency.

The medical devices 102, 104 shown in FIGS. 2 and 3 are only examples of a medical device. In some examples described herein, the medical devices 102, 104 are portable devices. In other examples, the medical devices 102, 104 are non-portable devices, such as computing devices like workstations. All different types of medical devices used to collect patient data can be used. Many configurations are possible.

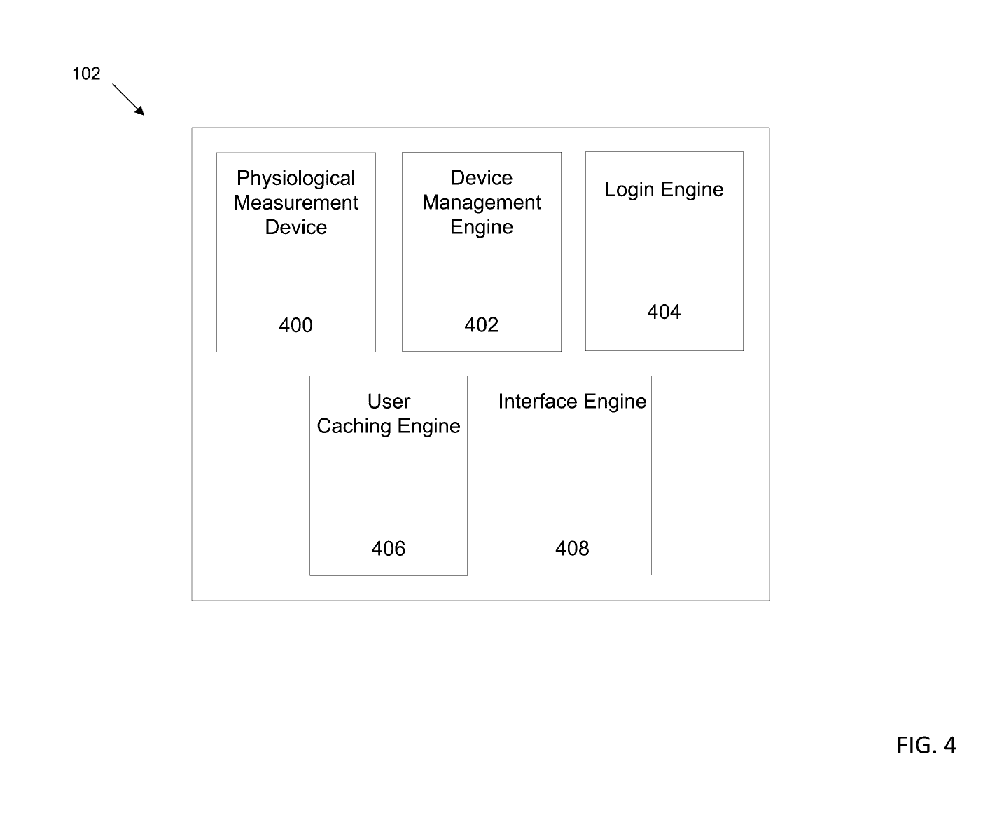

FIG. 4 is a block diagram illustrating example logical components of the medical device 102. The medical device 102 includes a physiological measurement device 400, a login engine 404, a user caching engine 406, and an interface engine 408.

The physiological measurement device 400 operates to measure one or more physiological parameters of a patient. Some embodiments include multiple physiological measurement devices.

An example physiological measurement device is an SpO2 measurement device. The SpO2 measurement device is designed to measure oxygen content within the blood of a patient. In some embodiments, the SpO2 measurement device includes a clip that attaches to an appendage of a patient, such as a finger. The clip is designed to detect and measure a pulse and an oxygen content of blood flowing within the patient.

Another example physiological measurement device is a non-invasive blood pressure (NIBP) measurement device. The NIBP measurement device is designed to measure blood pressure of a patient. In some embodiments, the NIBP device includes an inflatable cuff that attaches to an appendage of a patient, such as an upper arm of the patient. The inflatable cuff is designed to measure the systolic and diastolic blood pressure of the patient, the mean arterial pressure (MAP) of the patient, and the pulse rate of blood flowing within the patient.

Another example physiological measurement device is a temperature measurement device. The temperature measurement device is designed to measure the body temperature of a patient. In some embodiments, the temperature measurement device includes a handle and a temperature probe. The probe is designed to make physical contact with a patient in order to sense a body temperature of the patient. In some embodiments, the temperature probe is removable.

The device management engine 402 operates to manage the physiological measurement device 400. In some embodiments, the device management engine 402 transmits various control signals to the physiological measurement device 400 to activate or deactivate the physiological measurement device 400. Additionally, in some embodiments, the device management engine 402 operates to receive data captured by the physiological measurement device 400 and cause the received data to be stored. In some embodiments, the device management engine 402 transmits data captured by the physiological measurement device 400 to the server device 108 using a network interface to communicate over the network 106. In addition to the data captured by the physiological measurement device 400, some embodiments transmit identification information associated with a patient or a user who is operating the medical device 102. In some embodiments, the device management engine 402 is integral with the physiological measurement device 400.

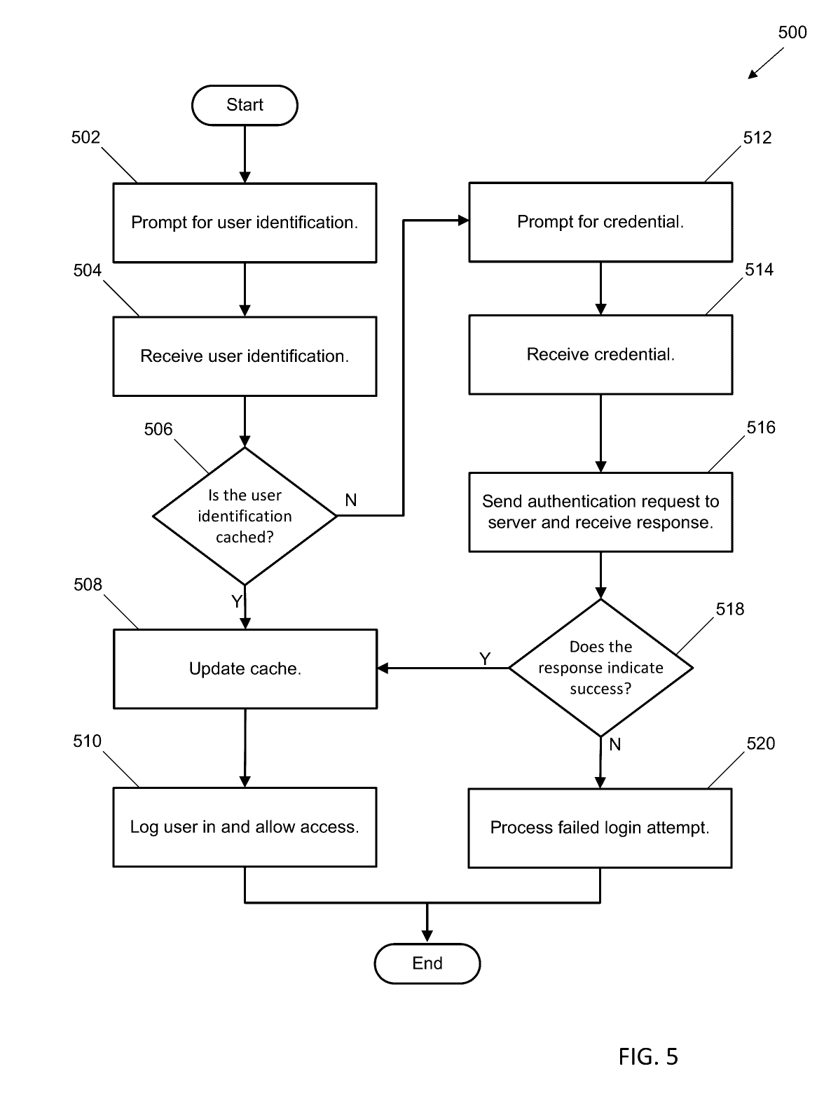

The login engine 404 operates to log a user such as a caregiver into the medical device 102. In some embodiments, the login engine 404 operates in conjunction with the user caching engine 406 to determine whether it is necessary to authenticate a user during a login. The login engine 404 is described in greater detail throughout, including with respect to FIGS. 5-7 and 9-11.

The user caching engine 406 operates to cache user information. The user caching engine 406 is described in greater detail throughout, including with respect to FIGS. 5-7 and 9-11.

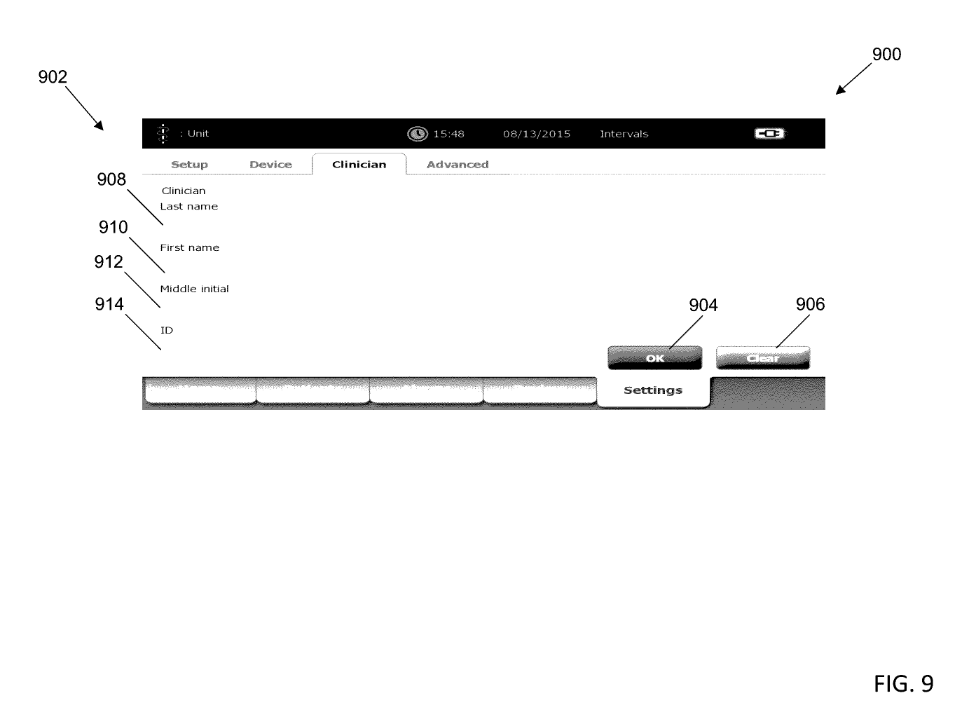

The interface engine 408 operates to generate a user interface for display on the display screen and to receive inputs from users. In some embodiments, the interface engine 408 generates user interfaces to prompt users for login information. The interface engine 408 may also generate user interfaces that display various information about the patient, including data related to physiological measurements captured by the physiological measurement device 400. Additionally, some embodiments display additional information about the patient that is received from the server device 108, such as previously captured physiological measurement values. Example user interfaces generated by the interface engine 408 are described and illustrated at least with respect to FIGS. 9-11.

Referring now to FIG. 5, an example process 500 performed by some embodiments of the medical devices 102, 104 is shown. The process operates to log a user in and allow the user to access the medical device. In some embodiments, operations of the process 500 are performed by the login engine 404 and/or the user caching engine 406.

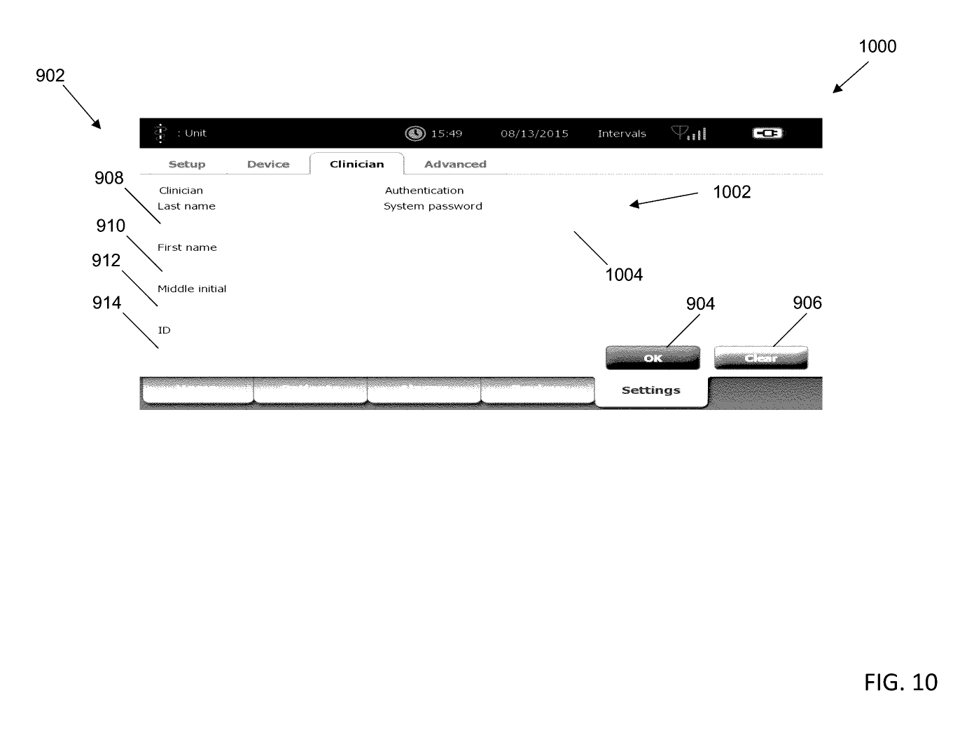

At operation 502, the user is prompted for user identification information. In some embodiments, the user interface engine generates a user interface to prompt the user for the user identification information. In some embodiments, the user identification information comprises a character string such as a username or identification number. Additionally, the user identification information may include multiple character strings (such as character strings a first name and a last name). In some embodiments, the user enters the user information using a physical keyboard or a virtual keyboard that is displayed on a touch-sensitive display screen. Additionally, in some embodiments, the user enters the user interface by presenting an identification object such as an identification card or employee badge to a reader device. The identification object may include an optical identifier such as a bar code or Quick Response (QR) code or a near-field communication identification technology such as a radio-frequency identification (RFID) tag. Example user interfaces generated by the interface engine 408 for prompting a user to enter user identification information are illustrated and described with respect to at least FIGS. 9 and 10.

At operation 504, the user identification information is received. In some embodiments, the user identification information is received when the user actuates a user-actuatable element on the user interface, such as a submit button. Additionally, in some embodiments, the user identification information is received as keystrokes are entered by the user or when the user changes focus on the user interface from a field for receiving user information to another field such as a credential entry field. Additionally, the user information may be received when the user successfully scans an identification object with the appropriate reader device.

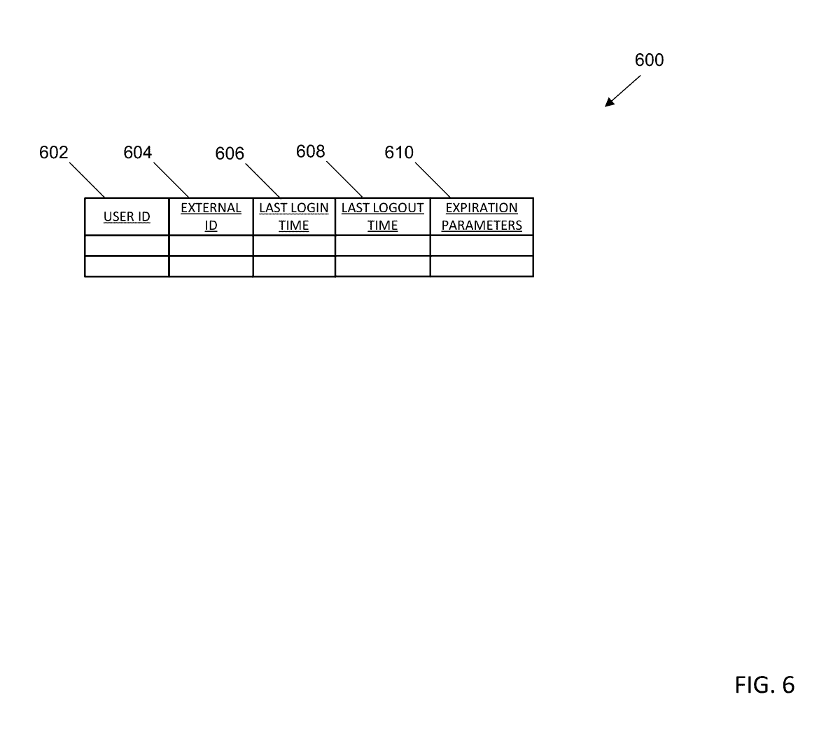

At operation 506, it is determined whether the entered user identification is cached. In some embodiments, determining whether the user identification is cached comprises executing a query against a database of cached user information. FIG. 6 provides an example table 600 of cache records for user information. The example table 600 includes a column 602 for storing user identification information, a column 604 for storing an external identifier, a column 606 for storing a last login time, a column 608 for storing a last logout time, and a column 610 for storing expiration parameters. In some embodiments, the table 600 is stored in a database such as a relational database (which may include other columns such as a primary key identifier column for establishing relational links). In some embodiments, the table 600 may be stored as a file in file systems. In at least some embodiments, the table 600 is stored in non-volatile memory and can thus be retained when the medical device 102, 104 is powered off. Each cached user may be associated with a cache record that includes values for some or all of the columns in the table 600.

In some embodiments, the table 600 includes additional, different, or fewer columns. For example, some embodiments may store one or more of an expiration time column, a number of remaining logins column, and a password column. Additionally, although each of the columns is shown as a single column, in some embodiments, multiple columns are used or foreign keys are stored that link a column to another table containing one or more tables are used. For example, the column 602 may be a link in a relational database to another table that stores more complex user information in multiple columns.

Returning now to operation 506 of FIG. 5, in some embodiments, determining whether the entered user identification is cached also comprises confirming that the cached user identification information has not expired (e.g., by evaluating the last login time or last logout time against the expiration parameters). In other embodiments, a maintenance process is relied on to clear out the user identification cache and it assumed that if a record appears in the cache, the record has not expired. Further, in some embodiments, determining whether the entered user information is cached comprises querying the server device 108 to confirm the account associated with the user has not been disabled even when the user information in the cached has not expired. If the user identification is cached, the process 500 continues on to operation 506. If not, the process 500 continues to operation 512.

At operation 508, the cache is updated to reflect that user has logged in again. For example, in some embodiments, the last login time is updated to the current time. Additionally, in some embodiments, the last logout time is cleared (e.g., to indicate that the user has not logged out) or updated to the current time (e.g., because the last logout time cannot be any earlier than the current time).

At operation 510, the user is logged in and allowed to access the capabilities of the medical device 102, 104. In some embodiments, operation 510 represents the end of the process 500 if the user information is cached.

As noted above, if the user identification is not cached, the process 500 continuous to operation 512. At operation 512, the user is prompted to enter a credential. In some embodiments, the credential is a password and the user is prompted to enter a character string representing a password.

At operation 514, the credential is received. The credential may be received when the user actuates a user-actuatable element on the user interface such as a submit button.

At operation 516, an authentication request is sent to the server device 108 and a response is received from the server device 108. The authentication request may be transmitted to the server device 108 using the network 106. In some embodiments, the authentication request may comprise the user identification and the credential. Additionally, in some embodiments, one or both of the user identification and the credential are encrypted before being transmitted to the server device 108. Although operation 516 has been described in terms of transmitting a request to a server, in some embodiments the authentication process is performed locally on the medical device and an authentication request is not sent to a server over the network.

At operation 518, it is determined whether the response indicates that the authentication attempt was successful. If the response indicates success, the process 500 continues to operation 508 where the cache is updated. In some embodiments, the response includes additional information that may also be added to the cache such as a username or identification for another system (e.g., a health information system). If instead, the response indicates that the authentication attempt was unsuccessful, the process 500 continues to operation 520.

At operation 520, the failed login attempt is processed. In some embodiments, the medical device 102, 104 is configured to perform a locking operation after a pre-determined number of consecutive failed login attempts. In some embodiments, the locking operation prevents further login attempts for a predetermined duration of time or until an administrator performs an unlock operation. For example, in some embodiments, the medical device 102, 104 will lock for five minutes after three unsuccessful login attempts. Alternatively, the medical device 102, 104 may lock for a shorter or longer time period, such as one minute, two minutes, ten minutes, fifteen minutes, thirty minutes, or one hour. Additionally, some embodiments, lock after fewer or more unsuccessful login attempts, such as two attempts, four attempts, five attempts, or ten attempts. In some embodiments, the medical device 102, 104 is locked preventing any other login attempts. Additionally, in some embodiments, an account associated with the entered user information is locked.

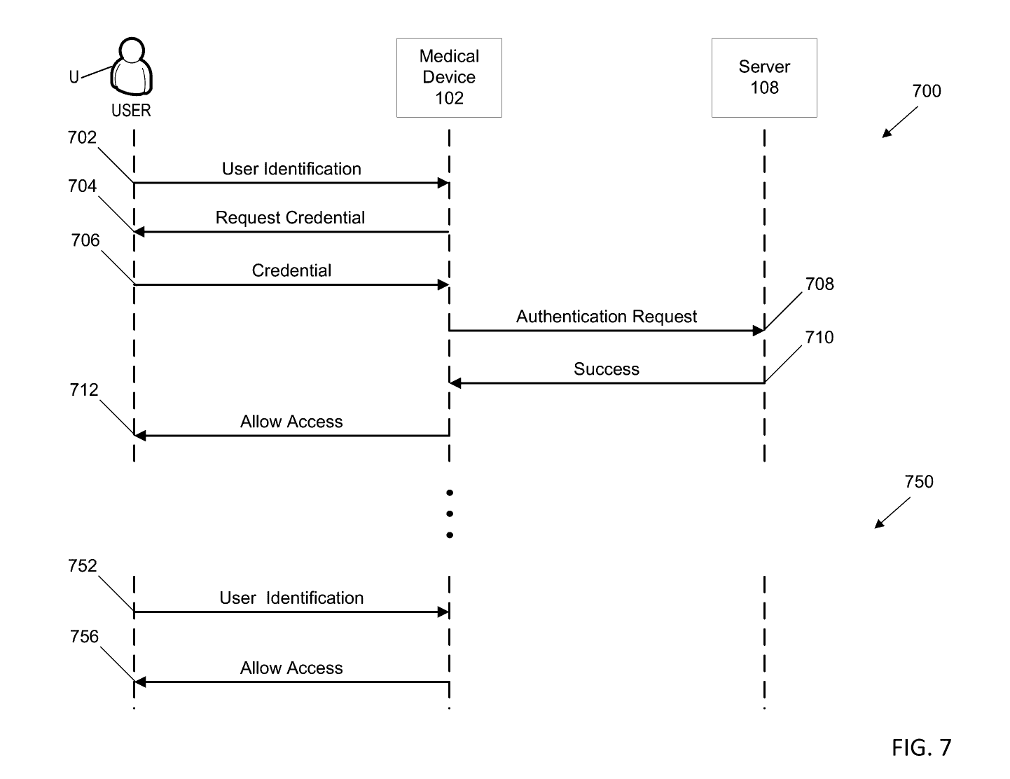

FIG. 7 is an example communication diagram between a user U, the medical device 102, and the server device 108. FIG. 7 includes a first example login attempt 700 in which the user information is not cached and a second example login attempt 750 in which the user information is cached.

In the first example login attempt 700, communication 702 from the user U to the medical device 102 includes a user identification. In some embodiments, upon receiving the user identification, the medical device 102 checks whether the user identification is cached. In this example login attempt 700, the user identification is not cached. Accordingly, a communication 704 from the medical device 102 to the user U requests a credential. For example, the communication 704 may be a prompt for a password. In response, a communication 706 from the user U to the medical device 102 includes a credential. A communication 708 from the medical device 102 to the server device 108 includes an authentication request, which may include the user identification and credential. A communication 710 from the server device 108 to the medical device 102 then indicates that the authentication was successful. In response, communication 712 from the medical device 102 to the user U allows the user to use and control the medical device. In some embodiments, the communication 712 comprises displaying a pop-up message or a user interface screen comprising controls to initiate measuring a physiological parameter.

In contrast to the first example login attempt 700, the second example login attempt 750 illustrates the communication that occurs when the user information is cached. The communication 752 from the user U to the medical device 102 includes the user identification. In this case, the user U has previously logged into the medical device 102 during the example login attempt 700 and so the user identification associated with the user U is cached by the medical device 102. After receiving the communication 752, the medical device 102 determines that the user information is cached. Accordingly, communication 754 from the medical device 102 to the user U allows the user to access to use and control the medical device. In some embodiments, the communication 754 is similar to the communication 712 which has been previously described. As illustrated in this second example login attempt 750, in at least some embodiments, when the user identification is cached, the medical device 102 does not communicate with the server device 108 before providing access to the user U.

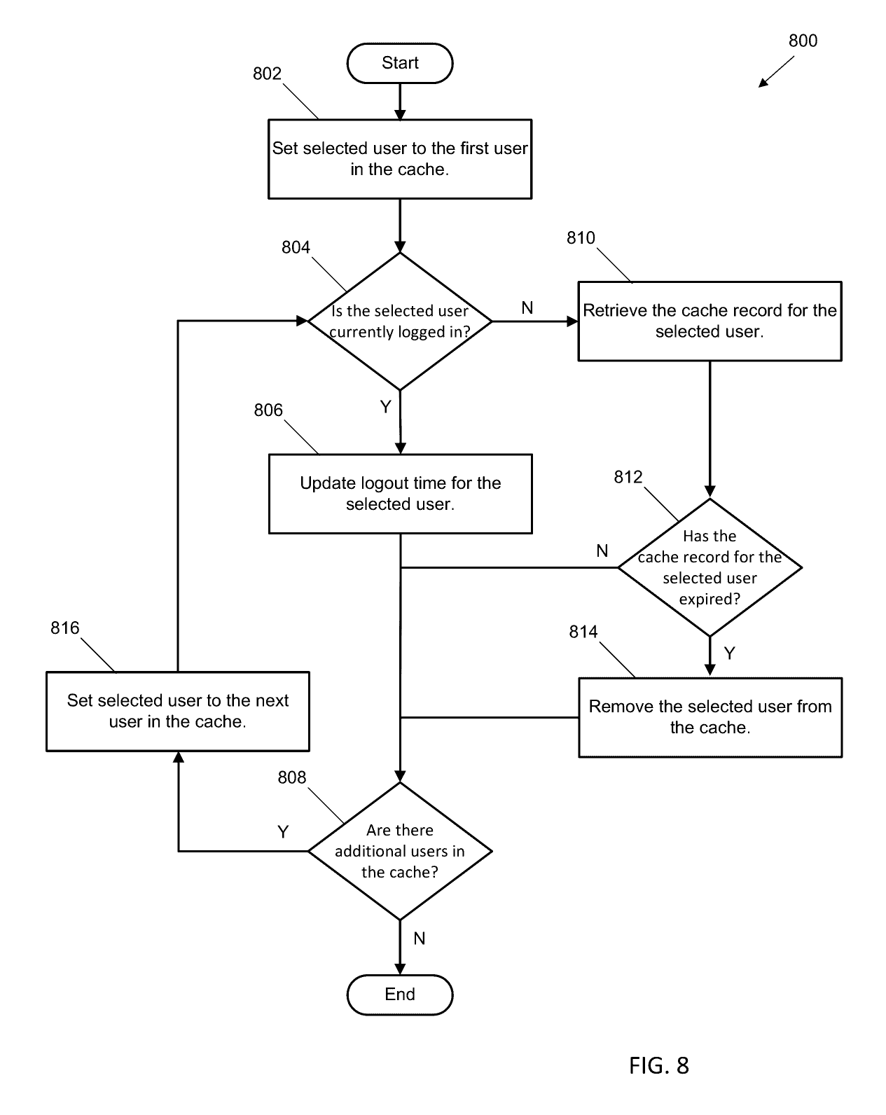

Referring now to FIG. 8, an example process 800 performed by some embodiments of the medical devices 102, 104 is shown. The process operates to maintain the user cache. In some embodiments, the process 800 is performed by the user caching engine 406.

At operation 802, the selected user is set to the first user in the cache. The selected user may be identified in order to process each of the users in the cache sequentially. In various embodiments, the first user is identified according to various criteria. For example, in some embodiments, the first user is identified based on when the users were added to the cache with the first user being the user that was added at the earliest time. In other embodiments, the first user may be identified based on an alphabetic ordering of the users by last name, first name, or a user identification string. Other orderings are used as well. Further in some embodiments, the users in the cache are ordered randomly in order to identify the first user.

At operation 804, it is determined whether the selected user is logged in. If yes, the process 800 continues to operation 806. If not, the process continues to operation 810.

At operation 806, the user logout time in the cache entry for the selected user is updated. For example, the user logout time may be cleared to indicate that the selects user is currently logged in. Alternatively, the user logout time may be set to the current time because the actual user logout time will be at least the current time.

At operation 808, it is determined whether there are additional users in the cache. If so, the process 800 continues to operation 816 where the selected user is set to the next user in the cache (e.g., based on the ordering used in operation 802). If not, the process 800 ends.

As not earlier, if the selected user is not currently logged in, the process 800 continues to operation 810. At operation 810, the cache record, including all of the fields of data, for the selected user is retrieved. In some embodiments, all of the data in the cache record is retrieved. In other embodiments, a subset of the stored data is retrieved. For example, the last logout time is retrieved in some embodiments. In other embodiments, the expiration parameters associated with the user are retrieved as well as the last login time or the last logout time.

At operation 812, it is determined whether the cache record for the selected user has expired. In some embodiments, determining whether the cache record has expired comprises evaluating the expiration parameters stored in the cache record. Additionally, in some embodiments, determining whether the cache record has expired comprises comparing the last logout time (or last login time) to the current time. If the cache record has expired, the process 800 continues on to operation 814. If not, the process 800 continues on to operation 808, where it is determined whether there are additional users in the cache.

At operation 814, the selected user is removed from the cache. The user may be removed from the cache by deleting the cache record. Alternatively, the user may be removed from the cache by setting a field (e.g., a deleted field) or clearing a field (e.g., an active field) in the cache record.

Referring now to FIG. 9, an example screen 900 for user logins generated by some embodiments of the interface engine 408 is shown. In some embodiments, the screen 900 is displayed by the display screen 200 of the medical devices 102, 104.

The screen 900 includes a user information panel 902, a first user-actuatable element 904, and a second user-actuatable element 906. Other embodiments of the screen 900 include fewer, additional, or different components.

The user information panel 902 operates to receive user identification information. In some embodiments, the user information panel 902 includes a last name field 908, a first name field 910, a middle initial field 912, and an identifier field 914. In some embodiments, the last name field 908, the first name field 910, the middle initial field 912, and the identifier field 914 are touch sensitive and are configured to receive textual input from a user. For example, upon touching one of these fields, a virtual keyboard may be displayed on the screen so that a textual input can be entered for the field. Additionally, in at least some embodiments, the identifier field 914 is configured to receive textural input from a user, while the last name field 908, the first name field 910, and the middle initial field 912 are configured to display information associated with the identifier received in the identifier field 914. This information that is displayed may be retrieved from the cache or it may be received from the server device 108. Further, some embodiments do not include one or more of the last name field 908, the first name field 910, and the middle initial field 912. Some embodiments of the user information panel 902 include fewer, additional, or different fields or components.

In some embodiments, the first user-actuatable element 904 is a button that may be activated by a touch or click using an input device of the medical device 102, 104. Upon activation, at least some of the information entered in the user information panel 902 is used to login a user. For example, in some embodiments, the process 500 is performed to login the user.

Referring now to FIG. 10, another example screen 1000 for user logins generated by some embodiments of the interface engine 408 is shown. In some embodiments, the screen 1000 is displayed by the display screen 200 of the medical devices 102, 104.

The screen 1000 includes the user information panel 902, the first user-actuatable element 904, the second user-actuatable element 906, and a credential panel 1002. The user information panel 902, the first user-actuatable element 904, and the second user-actuatable element 906 have been described previously at least with respect to FIG. 9. Other embodiments of the screen 1000 include fewer, additional, or different components.

The credential panel 1002 operates to receive credential information from a user. For example, the received credential information may be transmitted to the server device 108 to authenticate the user. In some embodiments, the credential panel 1002 includes a password field 1004. The password field 1004 operates to receive a textual input usable to authenticate the user. Some embodiments of the credential panel 1002 include fewer, additional, or different fields or components.

In some embodiments, the credential panel 1002 is not displayed until after it is determined that the user associated with the user information entered in the user information panel 902 is not cached. Alternatively, in some embodiments, the credential panel 1002 is displayed initially and upon determining that the user associated with the user information entered in the user information panel 902 is cached, fields in the credential panels are shown as being automatically entered (e.g., the fields are filled with asterisks). Further, in some embodiments, upon determining that the user associated with the user information entered in the user information panel 902 is cached and filling in the fields in the credential panel 1002, the interface engine 408 pauses for a predetermined period of time (e.g., one second) to emulate the process of submitting the login information to the server device 108. Additionally, in some embodiments, one or more of the fields in the user information panel 902 are populated when it is determined that the user is associated with a cache record (e.g., the identifier field 914 may be automatically filled based on determining that the data entered in the last name field 908 is associated with a cache record).

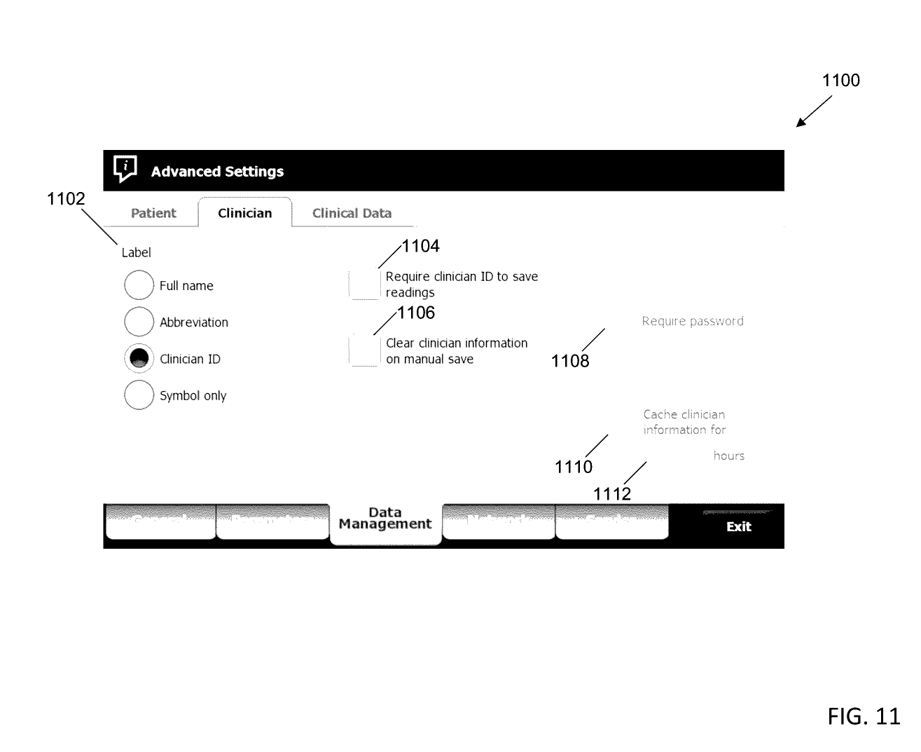

Referring now to FIG. 11, an example screen 1100 for user login settings generated by some embodiments of the interface engine 408 is shown. In some embodiments, the screen 1100 is displayed by the display screen 200 of the medical devices 102, 104. The screen 1100 may be displayed to allow a user (such as an administrator) to configure the medical devices 102, 104.

The screen 1100 includes a label selector control 1102, a require identifier checkbox 1104, a clear identifier on save checkbox 1106, a require credential checkbox 1108, a cache user information checkbox 1110, and a cache duration input 1112. Other embodiments of the screen 1100 include fewer, additional, or different components. For example, some embodiments may include other user interface controls that operate to select between using local and remote authentication. Although FIG. 11 illustrates a user interface for configuring various settings, some embodiments additionally or alternatively include other means of configuring at least some of these settings (e.g., a settings file that may be edited, a registry system to store settings, a database table of settings, various commands that may be entered in a terminal or elsewhere, an application programming interface, etc.).

The label selector control 1102 is configured to indicate and receive a user selection regarding how to display information about the user who is logged in. The information may be displayed across the top of the display screen 200 for example. In the example shown, the label selector control 1102 is a set of radio buttons in which each radio button is associated with an option and only one option may be selected at a time. In other embodiments, the options may be presented using different user interface elements such as a drop down list. Example options include “Full name,” “Abbreviation,” “Clinician ID,” and “Symbol only.” In some embodiments, when “Symbol only” is selected a set of asterisks will be displayed to indicate that a user is logged in without revealing any information related to the identity of the user.

The require identifier checkbox 1104 is configured to indicate and update the value of a setting that controls whether a user is required to provide identification before capturing and saving patient readings. The require identifier checkbox 1104 may toggle values (e.g., between cleared and set) when actuated. In some environments, it may be desirable to not require users to login or authenticate before capturing and saving patient readings.

The clear identifier on save checkbox 1106 is configured to indicate and update the value of a setting that controls whether the user identifier is cleared (e.g., by logging the user out) upon manually saving data (e.g., physiological measurements from patients). In some embodiments, the act of manually saving indicates that the user has completed using the medical device 102, 104. For example, if the user is a caregiver performing rounds, the user may capture measurements from the patient and then provide care to the patient without needing to use the medical device 102, 104 again until the user moves to the next patient.

The require credential checkbox 1108 is configured to indicate and update the value of a setting that controls whether the user is required to provide a credential to login. For example, the credential may be a password. In some embodiments, when this setting is active, the medical devices 102, 104 perform authentication with the server device 108 (at least when the user is not stored in the cache). In some embodiments, the require credential checkbox 1108 is inactive when the require identifier checkbox 1104 is cleared (i.e., not set).

The cache user information checkbox 1110 is configured to indicate and update the value of a setting that controls whether user information is cached on the medical devices 102, 104. Caching user information is described in greater detail throughout. In some embodiments, the cache user information checkbox 1110 is inactive when one or more of the require identifier checkbox 1104 and the require credential checkbox 1108 are cleared (i.e., not set).

The cache duration input 1112 is configured to indicate the value of and to receive updates to the value of a cache duration setting. In some embodiments, the cache duration setting controls how much time may elapse before a cache record is cleared from the cache (or otherwise considered invalid). In some embodiments, the cache duration setting operates as a default value that is applied when a duration value (or other expiration parameter) has not been set for a particular cache record.

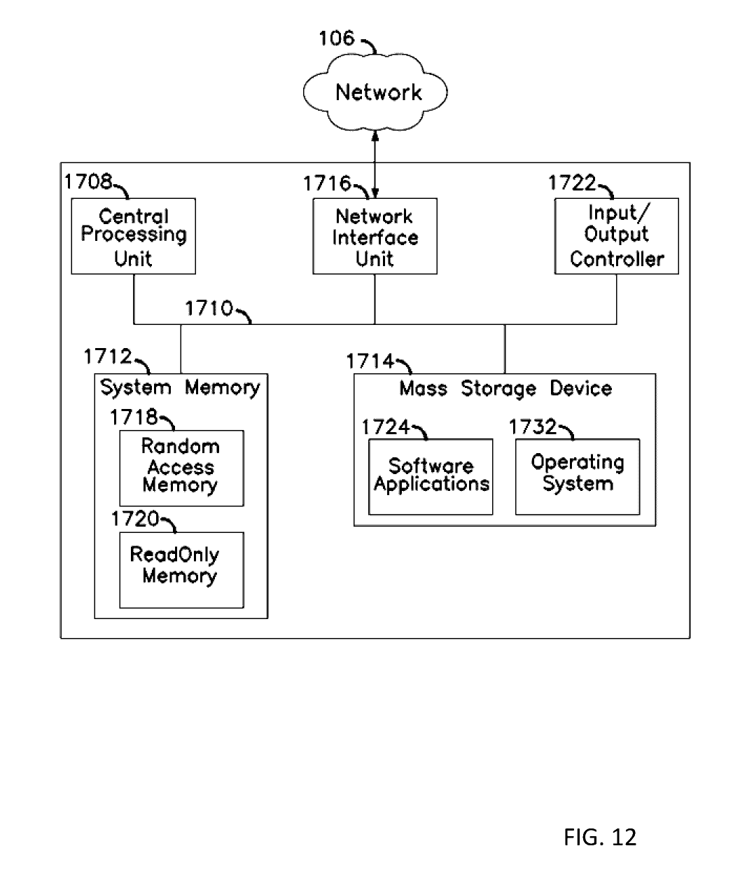

FIG. 12 illustrates example physical components of a computing device, such as the medical devices 102, 104. As illustrated, the device includes at least one central processing unit (“CPU”) 1708, a system memory 1712, and a system bus 1710 that couples the system memory 1712 to the CPU 1708. The system memory 1712 includes a random access memory (“RAM”) 1718 and a read-only memory (“ROM”) 1720. A basic input/output system containing the basic routines that help to transfer information between elements within the device, such as during startup, is stored in the ROM 1720. The device further includes a mass storage device 1714. The mass storage device 1714 is able to store software instructions and data.

The mass storage device 1714 is connected to the CPU 1708 through a mass storage controller (not shown) connected to the system bus 1710. The mass storage device 1714 and its associated computer-readable data storage media provide non-volatile, non-transitory storage for the device. Although the description of computer-readable data storage media contained herein refers to a mass storage device, such as a hard disk or CD-ROM drive, it should be appreciated by those skilled in the art that computer-readable data storage media can be any available non-transitory, physical device or article of manufacture from which the device can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the device.

According to various embodiments of the invention, the device may operate in a networked environment using logical connections to remote network devices through the network 106, such as a local network, the Internet, or another type of network. The device connects to the network 106 through a network interface unit 1716 connected to the system bus 1710. The network interface unit 1716 may also be utilized to connect to other types of networks and remote computing systems. The device also includes an input/output controller 1722 for receiving and processing input from a number of other devices, including a keyboard, a mouse, a touch user interface display screen, or another type of input device. Similarly, the input/output controller 1722 may provide output to a touch user interface display screen, a printer, or other type of output device.

As mentioned above, the mass storage device 1714 and the RAM 1718 of the device can store software instructions and data. The software instructions include an operating system 1732 suitable for controlling the operation of the device. The mass storage device 1714 and/or the RAM 1718 also store software instructions, that when executed by the CPU 1708, cause the device to provide the functionality of the device discussed in this document. For example, the mass storage device 1714 and/or the RAM 1718 can store software instructions that, when executed by the CPU 1708, cause the physiological monitor device to display the home screen and other screens.

Embodiments of the present invention may be utilized in various distributed computing environments where tasks are performed by remote processing devices that are linked through a communications network in a distributed computing environment.

The block diagrams depicted herein are just examples. There may be many variations to these diagrams described therein without departing from the spirit of the disclosure. For instance, components may be added, deleted or modified.

While embodiments have been described, it will be understood that those skilled in the art, both now and in the future, may make various improvements and enhancements.

What is claimed is:

1. A medical device, comprising:
a physiological measurement device configured to measure one or more physiological measurements from a patient;
a device management engine configured to receive data captured by the physiological measurement device;
a user caching engine configured to store cache records associated with users in a user cache; and
a login engine configured to:
receive a user identifier associated with a user;
determine whether the user identifier is associated with a cache record stored in the user cache; and
when determined that the user identifier is associated with a cache record stored in the user cache, log the user in.

2. The medical device of claim 1, wherein the medical device requires the user to login to save physiological measurements from patients.

3. The medical device of claim 2, wherein the login engine is further configured to, when determined that the user identifier is associated with a cache record stored in the user cache, update the cache record associated with the user identifier.

4. The medical device of claim 3, wherein updating the cache record comprises updating a last login time.

5. The medical device of claim 3, wherein updating the cache record comprises updating a last logout time.

6. The medical device of claim 1, wherein the login engine is further configured to when determined that the user identifier is associated with a cache record stored in the user cache:
determine whether the cache record has expired; and
when determined that the cache record has expired, prompt the user for a credential.

7. The medical device of claim 6, wherein determining whether the cache record has expired comprises comparing information stored in the cache record to a cache duration setting.

8. The medical device of claim 6, wherein determining whether the cache record has expired comprises evaluating expiration parameters associated with the cache record.

9. The medical device of claim 1, wherein the login engine is further configured to when determined that the user identifier is not cached:
prompt the user for a credential;
send an authentication request to a server device;
receive a response to the authentication request; and
when determined that the authentication request was successful, add a record associated with the user to the user cache.

10. The medical device of claim 9, wherein the credential is a password.

11. The medical device of claim 1, wherein the user caching engine is further configured to:
evaluate a cache record stored in the user cache to determine whether the cache record has expired; and
when determined that the cache record has expired, remove the cache record from the user cache.

12. The medical device of claim 1, further comprising a reader device configured to read an identification object associated with the user to receive a user identifier.

13. The medical device of claim 12, wherein the identification object is a badge comprising a barcode tag and the reader device is a barcode reader.

14. The medical device of claim 12, wherein the identification object is a badge comprising a radio frequency identifier tag and the reader device is a radio frequency identifier reader.

15. The medical device of claim 1, further comprising a user interface engine configured to:
prompt the user for a user identifier; and
when determined that the user identifier is associated with a cache record stored in the user cache, display a prompt for a credential, wherein the prompt is pre-filled; and
pause for a predetermined time period to emulates submitting an authentication request to a server.

16. A method of logging a user into a medical device, the method comprising:
receiving, by the medical device, a user identifier associated with the user;
determining whether the user identifier is associated with a valid cache record stored in the user cache;
when determined that the user identifier is associated with a valid cache record stored in the user cache, logging the user in; and
when determined that the user identifier is not associated with a valid cache record:
prompting the user for a credential;
determining if the user is authenticated based on the user identifier and credential;
when determined that the user is authenticated:
adding a record associated with the user to the user cache; and
logging the user in.

17. The method of claim 16, wherein determining whether the user identifier is associated with a valid cache record comprises evaluating information stored in the cache record to a cache duration setting.

18. The method of claim 16, further comprising when determined that the user identifier is associated with a valid cache record stored in the user cache, updating the cache record associated with the user identifier.

19. The method of claim 16, wherein determining if the user is authenticated comprises sending an authentication request to a server.

20. A medical device, comprising:
a physiological measurement device;
a device management engine configured to receive data captured by the physiological measurement device;
a user caching engine configured to store cache records associated with users in a user cache; and
a login engine configured to:
receive a user identifier associated with a user;
determine whether the user identifier is associated with an unexpired cache record stored in the user cache;
when determined that the user identifier is associated with an unexpired cache record stored in the user cache:
log the user in;
update a last login time of the cache record associated with the user identifier.
when not determined that the user identifier is associated with an unexpired cache record stored in the user cache:
prompt the user for a password;
send an authentication request to a server device;
receive a response to the authentication request; and
when determined that the authentication request was successful, add a record associated with the user to the user cache.
